SEQUENCE LISTING (11 sequences)

SEQ ID NO: 1 - protein, 10 aa, Artificial sequence
  KFF peptide
KFFKFFKFFK

SEQ ID NO: 2 - protein, 10 aa, Artificial sequence
  RFF peptide
RFFRFFRFFR

SEQ ID NO: 3 - protein, 23 aa, Artificial sequence
  Magainin 2
GIGKWLHSAKKFGKAFVGEIMNS

SEQ ID NO: 4 - protein, 21 aa, Artificial sequence
  Transportin 10
AGYLLGKINLKALAALAKKIL

SEQ ID NO: 5 - protein, 13 aa, Artificial sequence
  Indolicidin
ILPWKWPWWPWRR

SEQ ID NO: 6 - protein, 12 aa, Artificial sequence
  TAT peptide
GRKKRRQRRRPQ

SEQ ID NO: 7 - protein, 16 aa, Artificial sequence
  PENETRATIN 1 peptide
RQIKIWFQNRRMKWKK

SEQ ID NO: 8 - protein, 18 aa, Artificial sequence
  amphipathic peptide
LLIILRRRIRKQAHAHSK

SEQ ID NO: 9 - protein, 8 aa, Artificial sequence
  cyclic d,l-alpha-peptide
KQRWLWLW

SEQ ID NO: 10 - protein, 8 aa, Artificial sequence
  cyclic d,l-alpha-peptide
RRKWLWLW

SEQ ID NO: 11 - protein, 6 aa, Artificial sequence
  cyclic d,l-alpha-peptide
KKLWLW

(19) United States
(12) Patent Application Publication
(10) Pub. No.: US 2016/0038528 A1
(43) Pub. Date: Feb. 11, 2016

(54) Rapidly Adaptable Nano Therapeutics for Treatment of Infectious Disease

(71) Applicant: TECHULON INC., Blacksbrug, VA (US)

(72) Inventors: Brett MALONE, Pearisburg, VA (US); Joshua BRYSON, Blacksburg, VA (US)

(73) Assignee: Techulon Inc., Blacksburg, VA (US)

(21) Appl. No.: 14/777,002

(22) PCT Filed: Mar. 14, 2014
(86) PCT No.: PCT/US14/28804

§ 371 Date: Sep. 15, 2015

Related U.S. Application Data
(60) Provisional application No. 61/786,912, filed on Mar. 15, 2013.

(51) Int. Cl. A61K 31/713; A61K 47/48

(52) U.S. Cl. CPC A61K 31/713; A61K 47/48176; A61K 47/48315; G06F 19/3493

(57) ABSTRACT

The invention relates to rapidly adaptable nanotherapeutics. The therapeutics are nucleic acid molecules, such as, RNA, DNA, or modified-DNA. The nucleic acid therapeutics are preferably administered as a nanoparticle composition, further containing one or more synthetic polymers. The therapeutics are rapidly adaptable because the identification and design of the polynucleotide sequence containing the therapeutic sequence is based upon rapid computer-implemented bioinformatics and nucleic acid synthesis protocols. The rapid adaptable protocols differ from traditional methods of antibiotic and antipathogenic drug development, which are slow and do not address drug resistance issues. Furthermore, the invention encompasses a facility with dedicated apparatus for practicing the invention in military theater or where emerging pathogenic threats are located. This facility may be mobile and transportable as a dedicated unit.

### GOVERNMENT INTEREST

This work is based in part by the Defense Advanced Research Project Agency under Phase I SBIR contract number W911QX-12-C-0072. The US government has certain rights to the invention.

### FIELD OF THE INVENTION

The field of the invention relates to bioinformatics, molecular biology, nanoparticles, and pharmaceuticals, and treatment of infectious agents. More particularly, the field of the invention relates to the design, synthesis, formulation, and delivery of nucleic acid therapeutics for known or unknown infectious or pathogenic agents. The invention also relates to consolidated, dedicated apparatus for streamlined processing of rapid protocols of the invention.

### BACKGROUND OF THE INVENTION

Drug resistant pathogens represent a growing and significant risk to the general worldwide population, recovery of hospitalized patients, wounded soldiers, and recovering civilians. For US Defense agencies, the impact is felt in reduced operational capacity, slower response rates, and higher cost to supporting medical facilities. Across the private sector, post-treatment hospital infections account for thousands of deaths per year and billions of dollars in additional costs to healthcare infrastructure, as described in Klevens, et al. Public Health Report 2007 March-April; 122(2):160-6. Prolonged infection and complications during healing are major problems that remain without solutions to overcome issues of drug resistance. Studies that uncover pathogen mechanisms for adaptability and resistance, such as described in Peleg et al. Clin Microbiol Rev. 2008, 538-82, underline this problem.

Pathogens adapt to antibiotics by self-selecting on a regular basis. Furthermore, many pathogens are resistant to multiple drugs. In addition, new threats emerge regularly from foreign military operating areas where immunity is not established or the pathogen is not properly identified. An example of resistant Acinetobacter calcoaceticus-baumannii (Acb) infection is described in Albrecht et al. J Am Coll Surg. 2006, 203, 546-50. New infectious agents can come from natural sources or engineered sources. In either case, when the infectious agent is pathogenic, there may not be any drug treatment in existence. Response cycles to unknown, rare, or emergent threats are slow. Current methods follow traditional lines of drug development, which are hampered by narrow focus and suffer from a lack of a standardized cost-effective platform. While new antibiotics are developed to control new strains of adapted bacteria, the development process used in these traditional approaches remains too lengthy and narrow to provide a rapid and broad solution to combat drug-resistance, emergent threats, and rare infectious agents (orphan bugs). Certainly, current approaches to therapeutic development do not provide a viable solution to killer infectious agents that may be suddenly released in an act of bioterrorism.

### Impact of Drug-Resistant Pathogens

Military Operations:

The disruption to military operations and effectiveness of personnel is measured in terms of cost as well as downtime for trained specialists. Extended downtime accounts for suffering, unnecessary cost, and reduced military effectiveness. In critical scenarios such as shipboard operations, the potential for infection puts crews at risk and places the fleet in a position of understaffing. Albrecht et al. J Am Coll Surg. 2006, 203, 546-50 described the impact of Acinetobacter calcoaceticus-baumannii (Acb) infection on burn patients. The data indicated a high level of morbidity and longer hospital stays. Furthermore, there was significant resistance to many antibiotics on the market. Operational effectiveness is impacted when soldiers are unable to return to duty in a timely manner. Given current reductions in budgets and draw-downs across the major military services, maintaining a high level of readiness is critical to effective defense strength.

Private Sector and VA Hospital Environments:

It is estimated that post treatment infections cost the healthcare industry billions of dollars annually. The cost to hospitals, insurance companies, and taxpayers increases dramatically when hospital patients contract infections, such as methicillin-resistant Staphylococcus aureus (MRSA), Acb, or other drug resistant pathogens. If not caught and treated swiftly enough, rare types of infections that are flesh-eating can result in amputation. Based on a study by Richard Shannon at Allegheny General in Pittsburgh (Am J Med Qual 2006 November-December; 21(6 Suppl):7S-16S), the average additional cost to the hospital for each case is over $15,000 per patient.

Threats and Bioterrorism:

A study by the RAND Corporation (Bennett, et al. RAND Corporation, 2001) cites the fact that “small yet technically competent nations will continue to enhance their military capabilities; some of these countries might see a chemical/biological capability as an advantage over potential adversaries.” Drug-resistant pathogens represent one possible avenue for bio-threats and current development times for therapeutics take years, which is an unacceptable time frame. The rate at which bio-threats can be generated is faster than the current rate of response. The impact of evolving, unknown threats ranges from disruptive to pandemic-level.

Current approaches to health and safety with respect to infectious agents focuses on two areas: 1) preventative measures in treatment facilities, such as protective clothing, quarantine, and cleaning protocols; and 2) incremental improvements to existing antibiotic platforms. In the case of preventative measures, simple activities such as protective apparatus and clinical protocols help reduce the risk of infection and exposure. However, incomplete or partial enforcement of these preventative measures often leads to or perpetuates the local evolution and presence of drug resistant bacteria. In the case of existing antibiotic platforms, the cycle time for generating a novel antibiotic drug to a resistant organism follows traditional practices which are too slow and cannot keep up with pathogen evolution or engineering. Moreover traditional approaches will not be successful in defending against new pathogens, engineered pathogens, and unknown pathogens.

Genes that encode for resistance to antibiotics, like the antibiotics themselves, have evolved to current form over millions of years (D'Costa, et al. 2011 Nature 477, 457-461). Antibiotic use alone increases selective pressure in a population of bacteria allowing resistant bacteria selection preference. Resistance towards antibiotics has become faster and more common. However, in recent years, a decline in new approved antibiotic drugs has occurred (see Donadio, et al. 2010, The Journal of Antibiotics 63, 423-430).

Widespread use of antibiotics in agriculture and medicine has led to the evolution of antibiotic resistant bacteria superstrains. Small molecule antibiotics are being developed and approved at rates insufficient to compensate for the rapid evolution of these pathogens.

Several intrinsically invasive methods have been utilized for the delivery of genetic material into cells. However, they often trigger a cytotoxic response and therefore the chief barrier to using nucleic acids for the development of specific treatments is safe, efficacious delivery of DNA. This is due in large part because the level of disruption to the host cell is directly related to the physical attributes and transmembrane pathway of the nucleic acid. Indeed, high cell viability becomes even more essential when transfection is carried out in sensitive cell types, in vivo or in ex vivo implanted cells. Traditional means of carrying DNA into cells rely on cell membrane disruption such as electroporation or infection with viruses. Both of these have their own positive attributes and drawbacks, but are generally not conducive to rapid high throughput screening of multiple DNA constructs. Nor are they conducive to large scale protein production because electroporation doesn't scale to larger formats and the presence of a virus complicates protein purification and safety issues. Chemical transfection methods provide a more favorable alternative to electroporation and viruses. The initial choice for nucleic acid delivery was utilizing lipid-based delivery systems, which encapsulate the deliverable gene into a liposomal structure and enter the cell via membrane disruption/diffusion and active uptake. These agents in many cases are very efficacious delivery vehicles, however they exhibit severe drawbacks including extensive gene up-regulation, cell membrane damage, and low transfection efficiency in postmitosis cells.

Researchers more recently have turned to cationic polymers to meet their transfection needs, including poly-L-lysine (PLL), polyethyleneimine (PEI), chitosan, and their derivatives. Nucleic acid delivery using these compounds relies on complexation driven by electrostatic interactions between the gene and the polycationic delivery agent. Polymer-DNA complexes condense into particles on the order of 60-120 nm in diameter, a size suitable for active endocytosis by mammalian cells. Polymers such as linear PEI and PLL give very high transfection in a variety of primary and cells both in vitro and in vivo. Toxicity is sometimes associated with these polymers due to the high membrane-disrupting charge they carry.

### BRIEF SUMMARY OF THE INVENTION

The present invention is a new approach to killing superstrains, engineered pathogens, rare (orphan) pathogens, and novel natural pathogens. The present invention, which encompasses a streamlined platform for Rapidly Adaptable Nano Therapeutics (RANT), is a new method of drug development. The apparatus for carrying out this new method of drug development may be housed in a mobile, unified facility for streamlined end-to-end processing.

The present invention has the advantage of advanced and rapid capability to provide a bioterror defense and deterrent across the global theater. This platform sends a clear defensive message to potential adversaries, since new threat therapies can be developed rapidly rather than take several years. The RANT platform capability for addressing the emerging threats of bioterrorism represents a complete end-to-end consolidated solution, rather than incremental improvements in segments of a solution. The unified workflow process produces new potential therapeutics with timing in the order of days to weeks. More preferably, in the order of hours to days. The invention is not limited to the therapeutic development time. It is to be understood that some development times may be longer or shorter than others. For example, some therapeutics may require weeks to months to make. Overall, the invention condenses the multi-year process of drug development into a much shorter time span.

An aspect of the invention relates to a platform solution for making new therapeutics to treat patients infected with drug resistant pathogens, rare pathogens, and/or unknown pathogens, including bacteria, viruses, fungi, protozoa, parasites, and other infectious agents. The invention integrates bioinformatics with rapid synthesis and effective targeted delivery for biotherapeutics. The active ingredient(s) in the biotherapeutics comprise nucleic acids, including, but not limited to RNA, DNA, siRNA, antisense RNA, ribozymes, and modified-DNA polymers.

In one embodiment of the invention, the active ingredient is combined with a delivery polymer. In another embodiment of the invention, the active ingredient is coupled to a targeting molecule. In one aspect of the invention, the targeting molecule comprises a peptide. The peptide may comprise a cell penetration peptide (CPP). The peptide may also comprise a receptor ligand or fragment thereof. Peptides utilized may have one or more functions to facilitate cell targeting and/or membrane permeation.

In one aspect of the invention, the delivery polymer is cationic. In another aspect of the invention, the delivery polymer comprises phosphonium ions and/or ammonium ions.

In another aspect of the invention, the active ingredient is combined with a synthetic block co-polymer. The block copolymer has a pH responsive block that facilitates nucleic acid release from the nanoparticle after cell uptake.

In another example of the invention, the active ingredient is combined with a delivery polymer, where the composition forms nanoparticles in solution. In a further embodiment, the nanoparticles are stable in serum and are less than about 300 nm in size.

In one embodiment of the invention, the polymers of the nanoparticle are intracellularly biodegradable.

In the present invention, the active ingredient for neutralizing pathogens is designed using genomic sequences and/or expressed sequences. Greater efficacy is achieved from the specificity provided by sequencing pathogens and designing inhibitors to drug-resistant strains and novel pathogens based upon the sequence information. The invention encompasses the following platform components: Nucleic acid sequencing capabilities; databases for identification and categorization of pathogen sequences, software for informatics analysis; nucleic acid synthesis capabilities; and nanoparticle polymer synthesis capabilities.

The invention also encompasses a dedicated housing with custom apparatus adapted towards the RANT protocols.

In order to reduce cost and cycle time for response to drug-resistant pathogens, the present invention integrates technologies from different disciplines to form a response capability that addresses multiple threats and rapid pathogen neutralization. Multiple areas of technology are integrated; including, informatics, polymeric nanomaterials, rapid synthesis, and cell targeting. Custom nanomaterials comprising synthetic polymers create a flexible and low-cost delivery vehicle, resulting in cost and cycle time reduction. Other nucleic acid delivery mechanisms such as naked DNA and viral vectors have drawbacks. Naked DNA is quickly degraded and may not reach intended target cell. Viral vectors pose risks of mutation causing further disease, and take weeks to grow up to volumes necessary for dosing.

Rapid pathogen sequencing and identification lead to significant reduction in cycle time for early detection of specific strains of bacteria and identification of novel and rare infectious agents. The present invention utilizes bioinformatics algorithms for pathogen identification and characterization based on sequence analysis and utilizes existing databases of known pathogen and bacterial strains.

The present invention utilizes a cationic polymer-based nanoparticle drug delivery platform that is flexible and adaptable to a diverse set of nucleic acid therapeutic modalities. In particular, the therapeutic nucleic acids of the invention can be delivered to extracellular pathogens in addition to intracellular pathogens by attaching targeting molecules to the nucelic acid. The ability to conjugate nucleic acids to peptides for membrane disruption of pathogens or cellular targeting provides specificity and reduces toxicity. The synthetic polymer delivery formulation is both safer than viral delivery and more targeted than naked DNA. Further it does not require cell growth as viral vector preparation does.

Block copolymers useful in the RANT platform have three distinct blocks that address different aspects of successful delivery. Referring to FIG. 6, block A binds to the negatively charged nucleic acid backbone. Block B forms a sterically-blocked hydrodynamic shell to eliminate serum protein aggregation. Block C provides a pH-sensitive moiety which responds to endosomal acidification and enables endosomal disruption for nucleic acid escape after uptake into mammalian cells.

### System Integration

While each of the individual technology areas described above have become more advanced over time, the selection of these particular technologies and the unified combination thereof into a solution platform has never been suggested or carried out. Each technology area is a self-contained discipline. The interdisciplinary nature of the present systems and methods is unprecedented.

An overview of the platform of the invention is illustrated in FIG. 1. Rapidly Adaptable Nano Therapeutic (RANT) platform. The streamlined RANT approach incorporates unified deployment of several step-containing components for rapidly addressing engineered threats, drug resistant threats and orphan threats. These components combine to make a streamlined process and streamlined apparatus that identifies a pathogen, discovers essential orthogonal knockdown sequences for said pathogen, establishes targeted entry into pathogen, synthesizes high fidelity polynucleotides coupled with suitable bacterial entry peptides for blockage and/or knockdown, packages the synthetic polynucleotide with a suitable delivery vehicle, and delivers the synthetic targeted nucleic acid to various tissues within the host. The pretherapeutic steps of this drug development process may take place on dedicated apparatus in a single housing.

In one embodiment of the invention, the delivery vehicle comprises a cationic block copolymer containing phosphonium or ammonium ionic groups as described in PCT/US12/42974, incorporated by reference herein. In another embodiment of the invention, the delivery polymers comprise glycoamidoamines as described in Tranter et al. Amer Soc Gene Cell Ther, December 2011, incorporated by reference herein; polyhydroxylamidoamines, dendritic macromolecules, carbohydrate-containing polyesters, as described in US20090105115, incorporated by reference herein; and US20090124534, incorporated by reference herein. In other embodiments of the invention, the nucleic acid delivery vehicle comprises a cationic polypeptide or cationic lipid.

Additional embodiments of the invention include methods of providing a therapeutic nucleic acid composition to treat disease in a host caused by novel pathogenic agents and/or drug resistant pathogenic agents comprising:
a) Identifying a pathogen as novel, wherein identification comprises obtaining nucleic acid sequence information from the pathogen and analyzing the sequence information with software;
b) Identifying sequences within said pathogen that cause disease and/or drug resistance in a host;
c) Synthesizing one or more nucleic acid inhibitors of one or more of the sequences that cause disease; wherein the nucleic acid inhibitors are orthogonal to host sequences;
d) Combining the nucleic acid inhibitors with phosphonium ion-containing synthetic polymers to form nanoparticles stable in the bloodstream of the host; wherein the therapeutic nucleic acid composition comprises the nanoparticle; and wherein the therapeutic nucleic acid composition reduces disease and/or drug resistance caused by the novel pathogenic agent in a host.

The methods include embodiments where the phosphonium ion-containing synthetic polymer is a block copolymer comprising a pH responsive block for intracellular release of nucleic acid; wherein the nucleic acid inhibitor is coupled to a peptide; wherein the peptide is a cell penetrating peptide; wherein the peptide is a host cell targeting peptide; wherein the pathogen has a cell membrane; and/or wherein the pathogen is bacterial, viral, protozoan, or parasite.

Additional embodiments of the invention include methods of providing a therapeutic nucleic acid composition to treat disease caused by a viral pathogen comprising:
a) Identifying a viral pathogen, wherein identification comprises obtaining nucleic acid sequence information from the pathogen and analyzing the sequence information with software;
b) Identifying sequences within said pathogen that cause disease in a host;
c) Synthesizing nucleic acid inhibitors of one or more sequences that cause disease; wherein the nucleic acid inhibitors are orthogonal to host sequences;
d) Combining the nucleic acid inhibitors with phosphonium ion-containing synthetic polymers to form nanoparticles stable in the bloodstream of a host; wherein the therapeutic nucleic acid composition comprises the nanoparticles; and wherein the therapeutic nucleic acid composition reduces disease caused by the viral pathogen in a host.

The methods include embodiments where the phosphonium ion-containing synthetic polymer is a block copolymer comprising a pH responsive block for intracellular release of nucleic acid; wherein the nucleic acid inhibitor is coupled to a peptide; wherein the peptide is a cell penetrating peptide; and/or wherein the peptide is a host cell targeting peptide.

Additional embodiments of the invention also include a bioterrorism response unit comprising:
a) A dedicated nucleic acid sequencer, wherein the dedicated sequencers feed output to a dedicated informatics system;
b) A dedicated informatics system comprising hardware and software for analyzing nucleic acid sequences from known and unknown pathogenic agents;
c) A dedicated nucleic acid synthesizer for synthesizing polynucleotides;
d) A dedicated formulation apparatus for making therapeutic nanoparticles;
e) A dedicated storage apparatus for storing doses of therapeutic nanoparticles.

The bioterrorism response unit comprises embodiments wherein the response unit is mobile and/or wherein the response unit is contained within a single housing.

### BRIEF DESCRIPTION OF THE DRAWINGS/FIGURES

FIG. 1 shows an overview of the platform system, where new informatics algorithms for identification and design are combined with low-cost nucleic acid synthesis and advanced delivery to provide a rapid capability to combat drug resistant and unknown pathogens.

FIG. 2 shows an example of an end-to-end solution provided by the invention, where a new pathogen is detected, its genetic material is sequenced, bioinformatics algorithms are utilized to design anti-pathogen nucleic acid-based therapeutics, and the therapeutic nucleic acid(s) are combined with a delivery polymer for administration. In this example RNA, siRNA, and peptide-conjugated RNA are included as nucleic acid therapeutic modalities, however, one of ordinary skill in the art will recognize that DNA and modified-DNA can be used in the invention.

FIG. 3 shows a flowchart of an embodiment of the bioinformatics software. Other software flowcharts are contemplated as part of the invention, wherein the software comprises at all or part of the functionalities described in this figure.

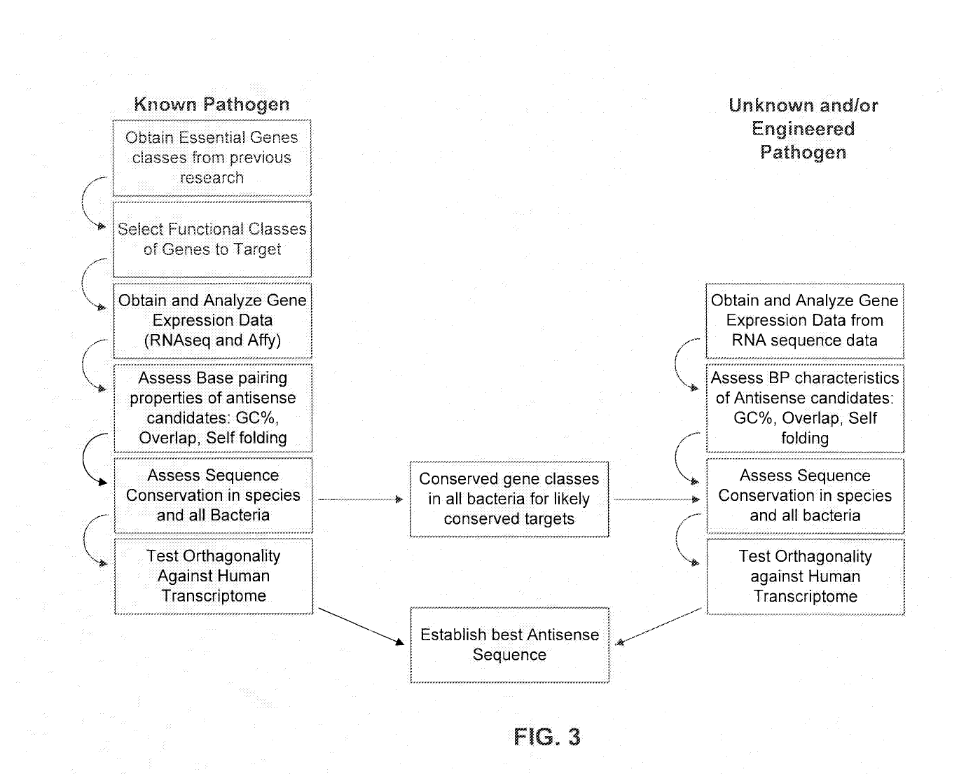

FIG. 4 shows an example of the platform of the invention where bacteria represents a disease-causing organism, and where bioinformatics is used to determine an antisense RNA therapeutic against bacterial sequences. Once the RNA therapeutic is synthesized, it is conjugated to a permeation peptide and optionally mixed with a delivery polymer. In this example, there is no delivery polymer.

FIG. 5 shows an example of the platform of the invention where an intracellular virus is a disease-causing organism, and where bioinformatics is used to determine an RNA therapeutic against viral sequences. Once the RNA therapeutic is synthesized, it is conjugated to a cell targeting peptide and then mixed with a delivery polymer to form a polyplex particle. The resulting nanoparticle is part of the delivery formulation. This example illustrates the pH responsive release of nucleic acid from polyplexes.

FIG. 6 shows the family of delivery polymers useful in the present invention, wherein n is a number ranging from 2 to 1,000. Block A is a complexation block which binds to negatively charged nucleic acids; Block B enables formation of a nanoparticle with a sterically-blocked hydrodynamic shell; and Block C provides a pH-sensitive moiety which responds to endosomal acidification. This figure depicts an embodiment where a Block A contains a phosphonium ion.

FIG. 7 depicts options for Block C, as referred to in FIG. 6, wherein n is a number ranging from 2 to 1,000.

FIG. 8 depicts options for Block B, as referred to in FIG. 6, wherein R is C1-24 alkyl; and n is a number ranging from 2-1,000.

FIG. 9 shows an example of bioinformatics infrastructure utilized in the present invention. The system includes hardware and software such as servers, data bases, data storage apparatus, interface services, web services, client interfaces and analytics software.

FIG. 10 shows an optional embodiment of the invention where the RANT system is within a single unified housing. In this embodiment, the end-to-end system is in a POD enclosure, wherein the POD is transportable, has inputs for water, electricity, inside environmental controls, lab set-up including but not limited to molecular biology equipment and reagents, at least part of informatics hardware/software, telecom network, nucleic acid synthesis apparatus, formulation apparatus, and storage for dosage forms of RANT therapeutics. Other optional embodiments include remote control of apparatus via telecom networks. In other embodiments, informatics hardware and software are housed separately, but are operatively linked to the POD via telecom.

### BRIEF DESCRIPTION OF THE SEQUENCE LISTING

The peptide sequences in the sequence listing represent peptides that target and/or localize nucleic acids and nanoparticles to bacterial cells and promote bacterial membrane permeation.

TABLE 1
Sequence Listing

| Peptide Name | Amino Acid Sequence |
|---|---|
| KFF peptide | KFFKFFKFFK (SEQ ID NO: 1) |
| RFF peptide | RFFRFFRFFR (SEQ ID NO: 2) |
| Magainin 2 | GIGKWLHSAKKFGKAFVGEIMNS (SEQ ID NO: 3) |
| Transportin 10 | AGYLLGKINLKALAALAKKIL (SEQ ID NO: 4) |
| Indolicidin | ILPWKWPWWPWRR (SEQ ID NO: 5) |
| TAT peptide | GRKKRRQRRRPQ (SEQ ID NO: 6) |
| PENETRATIN 1 peptide | RQIKIWFQNRRMKWKK (SEQ ID NO: 7) |
| amphipathic peptide | LLIILRRRIRKQAHAHSK (SEQ ID NO: 8) |
| cyclic d,1-alpha-peptide | KQRWLWLW (SEQ ID NO: 9) |
| cyclic d,1-alpha-peptide | RRKWLWLW (SEQ ID NO: 10) |
| cyclic d,1-alpha-peptide | KKLWLW (SEQ ID NO: 11) |

### DEFINITIONS

The terms used in this disclosure have ordinary meanings as used in the art.

A polymer is a linear chain of units called monomers. In a polymer, the monomeric units may be identical or they may be different. Polymers may be natural (made in nature) or may be synthetic. Polymers of the present invention comprise nucleic acid polymers, polypeptides, and synthetic delivery polymers.

A nucleic acid is a linear polymer of nucleotides. Nucleic acids made in nature contain deoxyribonucleotide (DNA) bases adenine, cytosine, guanine, and thymine; or ribonucleotide (RNA) bases adenine, cytosine, guanine, and uracil. As used herein, polynucleotide and oligonucleotide refer to a nucleic acid molecule and include genomic DNA, cDNA, RNA, mRNA of any length. Nucleic acid, polynucleotide, oligonucleotide are terms that may be used interchangeably.

Modified nucelic acids are non-natural polymers that hybridize to natural DNA and RNA. Examples of modified nucleic acids are phosphorothioate polynucleotides (PS-ODNs), locked nucleic acids (LNAs), 2′-O-methyloligoribonucleotides (2′O-Mes), phosphorodiamidate morpholino oligonucelitides (PMOs), peptide nucleic acids (PNAs). Modified nucleic acids are generally more resistant to degradation than natural nucleic acids.

The term antisense polynucleotide refers to a nucleic acid molecule that is complementary to at least a portion of a target nucleotide, sequence of interest and hybridizes to the target nucleotide sequence under physiological conditions. Antisense molecules specifically hybridize with one or more nucleic acids encoding a preselected target nucleic acid. The terms target nucleic acid and nucleic acid encoding the target encompass DNA encoding the target, RNA (including pre-mRNA and mRNA) transcribed from such DNA, and also cDNA derived from such RNA. The hybridization of an antisense compound with its target nucleic acid interferes with the normal function of the nucleic acid. This modulation of function of a target nucleic acid by compounds which specifically hybridize to it is generally referred to as antisense. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the target. In the context of the present invention, modulation means either an increase (stimulation) or a decrease (inhibition) in the expression of a gene. In the context of the present invention, inhibition is the preferred form of modulation of gene expression.

Polynucleotides are described as complementary to one another when hybridization occurs in an antiparallel configuration between two single-stranded polynucleotides.

Proteins are polymers containing one or more chains of amino acids bonded together by peptide bonds. Proteins typically fold into a three dimensional form, facilitating a biological function.

A polypeptide is a polymer of amino acids bonded together by peptide bonds. The terms protein and polypeptide and peptide are generally used interchangeably, although polypeptides and peptides are generally shorter in length than proteins.

The terms charged, uncharged, cationic and anionic refer to the predominant state of a chemical moiety at near-neutral pH, e.g. about 6 to 8. Preferably, the term refers to the predominant state of the chemical moiety at physiological pH, that is, about 7.4. Thus a cationic backbone linkage is predominantly positively charged at pH 7.4.

The terms pathogen and pathogenic agent refer to an organism capable of infecting and causing disease in a host, as well as producing infection-related symptoms in the infected host.

### DETAILED DESCRIPTION OF THE INVENTION

The present invention may be understood more readily by reference to the following detailed description of the embodiments of the invention and examples included herein. However, it is to be understood that this invention is not limited to specific nucleic acids, specific polypeptides, specific delivery polymers, specific cell types, specific host cells, or specific pathogens, etc. It is also to be understood that the terminology used herein is for the purpose of describing embodiments of the invention and is not intended to be limiting.

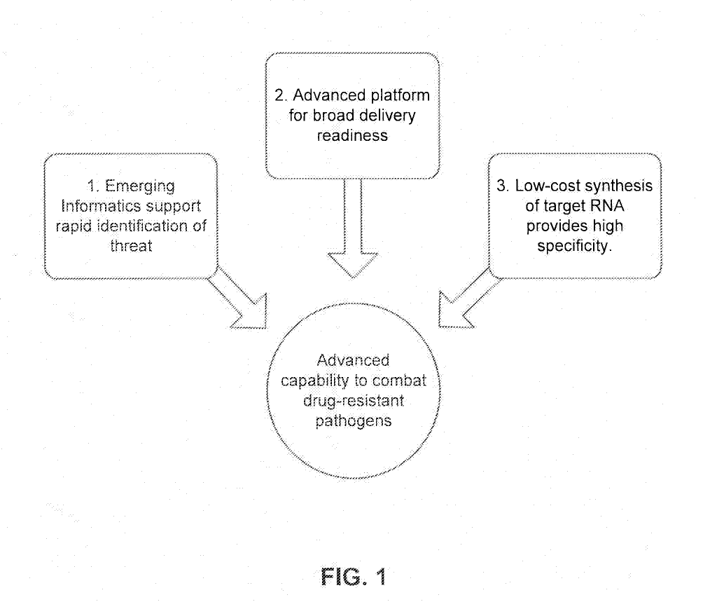

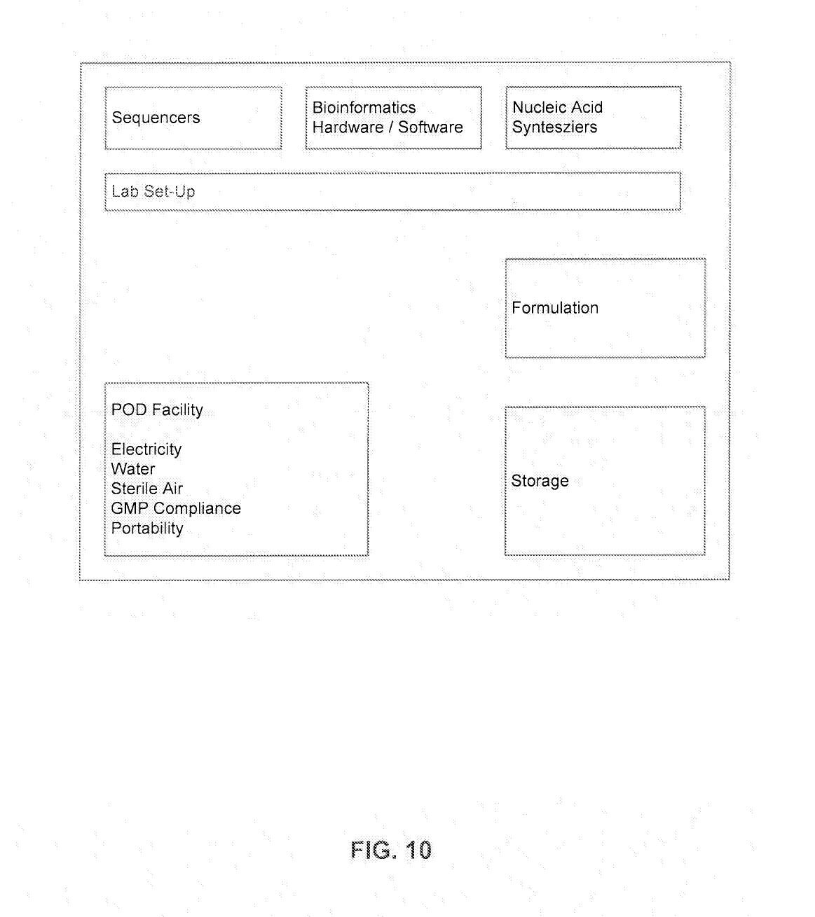

An embodiment of the end-to-end system of the invention is illustrated in FIG. 10. In this embodiment, the entire system is housed within a single unit, wherein the unit contains all necessary apparatus to carry out the inventive steps. Alternative apparatus configurations are also encompassed by the invention, wherein some components are housed separately. The platform system and apparatus combine nucleic acid sequencers and new informatics algorithms with nucleic acid synthesis and advanced delivery vehicles to provide rapid method of drug development with the capability to combat drug resistant pathogens, rare pathogens, and unknown pathogens. FIG. 1 represents an overview of the interdisciplinary technologies incorporated into a unified platform.

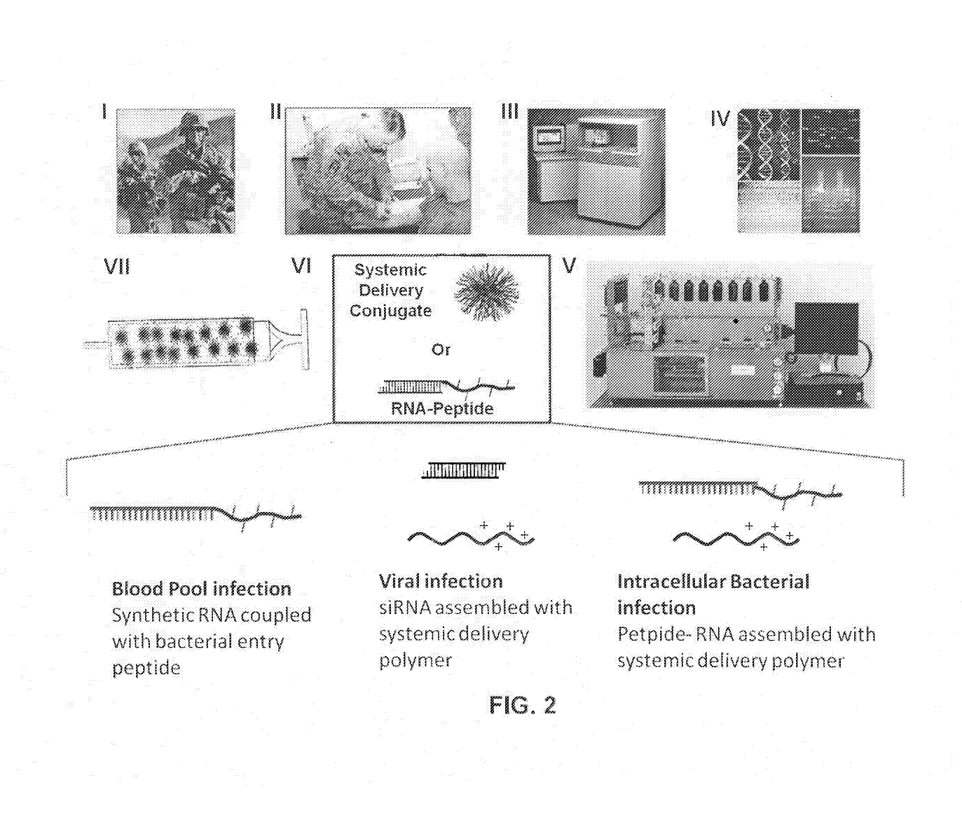

FIG. 2 shows an example of how the platform can be utilized for RNA-based therapeutics against a new bacterial pathogen. When a new pathogenic infectious agent is detected in a patient, such as a soldier, its genetic material is first sequenced. Then bioinformatics algorithms are utilized to categorize the pathogen and identify relevant genes for therapeutic knockdown, and to design anti-pathogen nucleic acid-based therapeutics. Once the therapeutic nucleic acid(s) are synthesized, they are combined with a delivery polymer for administration. The RANT workflow process encompasses these steps: 1: Injury and exposure. 2: Diagnostics and collection of samples. 3: Pathogen sequencing. 4: Bioinformatics to identify pathogen and establish best sequence for RNA blocking based on orthogonality, essential function, and lack of mutation. 5: Rapid high-fidelity synthesis of regulatory RNA conjugated to bacteria penetrating peptide. 6: Determination of whether to administer naked peptide-RNA for blood an extracellular threats or to use systemic delivery for intracellular threats. 7: Patient administration of delivery particle or peptide-RNA conjugate for blood infections.

In one embodiment of the invention, the delivery polymer is a cationic block copolymer that forms a nanoparticle when combined with nucleic acids, where the nanoparticle is suitable for administration. In a further embodiment, the polymers of the nanoparticle are sensitive to intracellular pH which improves intracellular nanoparticle release of the nucleic acid(s).

The RANT platform of the invention comprises one or more elements described in the following examples.

### EXAMPLE I

### Rapid Pathogen Identification

The invention comprises an informatics system, which contains workflow for sequencing and identification of pathogens, and inhibitor design based on sequence information. First the genetic material, either DNA or RNA, from a pathogen (bacteria, virus, fungus, parasite, etc.) is subject to sequencing. The genetic material may be genomic DNA, genomic RNA, or expressed sequences. With new sequencing technologies, microbial genomes can be sequenced in near real-time in a matter of hours (Torok J Antimicrob Chemother June 2012). It is desirable that the whole genome be sequenced; however, the invention encompasses scenarios where incomplete sequence information is available. The sequence information identifies the pathogen as known or unknown. Unknown pathogens are characterized by sequence similarities or other comparisons to known pathogens. From the sequence information of the known or unknown pathogen, a gene or set of genes for expression inhibition are identified and selected. Inhibitors, such as antisense RNA, siRNA, or DNA are then designed by the informatics algorithms.

This system comprises both nucleic acid sequencing and informatics. Informatics algorithms identify the sequence targets in genes whose suppression kills or disables the bacterium, virus, or other pathogen. Target genes can include essential genes, housekeeping genes, pathogenicity (invasion) genes, or other genes which have predictive value for organism inhibition. Sequence targets are also filtered against human transcript data to create an orthogonal therapeutic and avoid negative impact on patients. An orthogonal therapeutic is designed to have as little interaction with human sequences as possible and result in lower toxicity to the patient. The system handles a range of pathogenic organisms. For example, the system handles known bacteria as well as novel, culturable species. In addition, the system handles infective agents that are novel, not culturable, and for which incomplete genomic sequence is obtained from infected tissues.

A novel agent that lacks a close reference sequence is subject to rapid extensive sequencing of material isolated from the patient or wound. Sequence information from genomic DNA and expressed genes is desirable. A sample coming from a wound is likely to have a mixture of genomes, including human host and common bacteria. Host sequences and common bacterial sequences are removed from the data output to isolate the pathogen sequences. Sequence information from the infection shows which transcripts are being highly expressed in the pathogen. Highly expressed sequences are an indication of vulnerability to antisense suppression. The bioinformatics system of the invention handles both the reference genome and de novo sequence (including RNA transcripts) scenarios.

Therapeutic Design:

For therapeutic nucleic acid design, different categories of functions can be targeted for knockdown; such as, essential genes (essential to the viability of the pathogen), pathogenicity factors (to block ability to invade), and antibiotic resistance enzymes (to render resistant bacteria sensitive to traditional antibiotics). Essential genes have the advantage that they tend to be conserved across strains and species. In well-studied bacteria such as E. coli and Staphylococcus, essential genes have been tabulated (Forsyth et al. Mol. Microbiol. 2002 March; 43(6):1387-400; Gerdes, et al. J. Bacteriol 2003 October; 185(19):5673-84). These and other resources are part of the informatics databases of the invention. For example, Virginia Tech Bioinformatics Institute (VBI) has a vast resource of sequence databases. Other resources useful in the invention include National Center for Biotechnology Information (NCBI) databases, European Molecular Biology Laboratory (EMBL) databases, J Craig Venter Institute (JCVI) databases, and Genomic Sequencing Center for Infectious Disease (GSCID) databases.

High expression level is a hallmark of effective antisense targets. Only expressed genes can be suppressed and high mRNA expression signals a need for large amounts of product and/or rapid turnover by protein degradation leaving the organism vulnerable to a slowdown of synthesis. Experimental support for the link between expression levels and target validity is provided by microarray data in E. coli in relation to essential genes and the proven antisense target acpP. The NCBI Gene Expression Omnibus (GEO) yields 553 data sets on the Affymetrix E. coli Genome 2.0 Array spanning a range of genotypes and growth conditions. Normalizing these arrays in one analysis allows us to identify genes expressed at high levels across most or all conditions. Essential genes are significantly higher in expression level than non-essential genes, and the acpP gene has nearly the highest expression of all.

The target sequence selection system of the invention has data storage and analysis components. The system of the invention stores summaries of expression levels for genes of well-studied bacteria mined from sources such as the NCBI Gene Expression Omnibus (GEO). Consistently high expression levels across growth conditions is one factor in ranking genes as antisense targets. It is possible that a rare or novel infection arises which cannot be placed in a well-studied pathogen group and no expression data is available. If genomic sequence is available (from sequencing genomic DNA as opposed to mRNA), a characteristic of the gene-coding regions known as codon bias is used as a proxy indicator of highly expressed genes. There is a correlation between gene expression and a measure of codon bias, indicating that codon bias has the ability to predict highly expressed genes in the absence of expression data. Another criterion for target gene selection is lack of redundancy. Genes which are unique in the genome, both in terms of sequence similarity and annotated function, are considered better targets than genes with redundant copies or similar-functioning analogs. Furthermore, avoiding interactions with human gene expression is achieved by a database of known human transcripts to screen candidate antisense reagents against.

There is a relationship among expression, codon bias, and essentiality in microbial genes. Gene expression is normalized and averaged by gene across microarrays. Plots of microarray data show expression versus Codon Adaptation Index (CAI), a measure of codon bias, for all genes and essential genes. When the product of CAI and log (expression) are used, the level of essential gene enrichment is depicted. At composite scores over 6.0, genes are identified as essential. This type of analysis shows that target therapeutic genes are identified by informatics algorithms of the invention when novel pathogenic agents are encountered.

In summary, the gene target selection algorithm of the invention considers factors such as, essentiality; drug resistance; pathogenicity mechanism; high expression; codon bias; lack of redundancy at sequence or annotated function level; and avoidance of similarity to human transcripts.

After one or more target genes are identified and selected, further informatics software of the invention specifies the nucleotide sequence for the antisense or other inhibitory nucleic acid reagent. For antisense, this is typically a polynucleotide spanning the start codon. The software scans windows of size 12 bases (as a non-limiting example) including the start codon and ranks self-folding potential by base content. Selection of antisense sequence is finalized manually from these data or through an automated process derived from empirical data and parameter weighting. The informatics component of the RANT process is a predictive tool to design nucleic acids that disrupt pathogenesis and virulence.

Data Architecture and Design:

Database architecture is designed and implemented in MySQL, however, other database software and implementations may be utilized in the invention, as is understood by one skilled in the art. The functional interface that allows the user to input sequences, analyze a series of targets, and output reports to arrive at a conclusion regarding levels of match is computer implemented in software. For example, the invention comprises a system that permits the user to query pre-computed analysis results for a given sequence. This system has a front end for browsing and querying, and a back end database loaded with one or more sets of pre-computed results. Language and database configuration used in the invention are a Javascript based web front end and a MySQL back end; although one skilled in the art can utilize other programming languages and databases. Search analytics are performed using Perl scripts, although, other programming languages may be utilized.

In one aspect of the invention, the input and output from one step to another is automated. In another aspect of the invention, some steps are operated remotely.

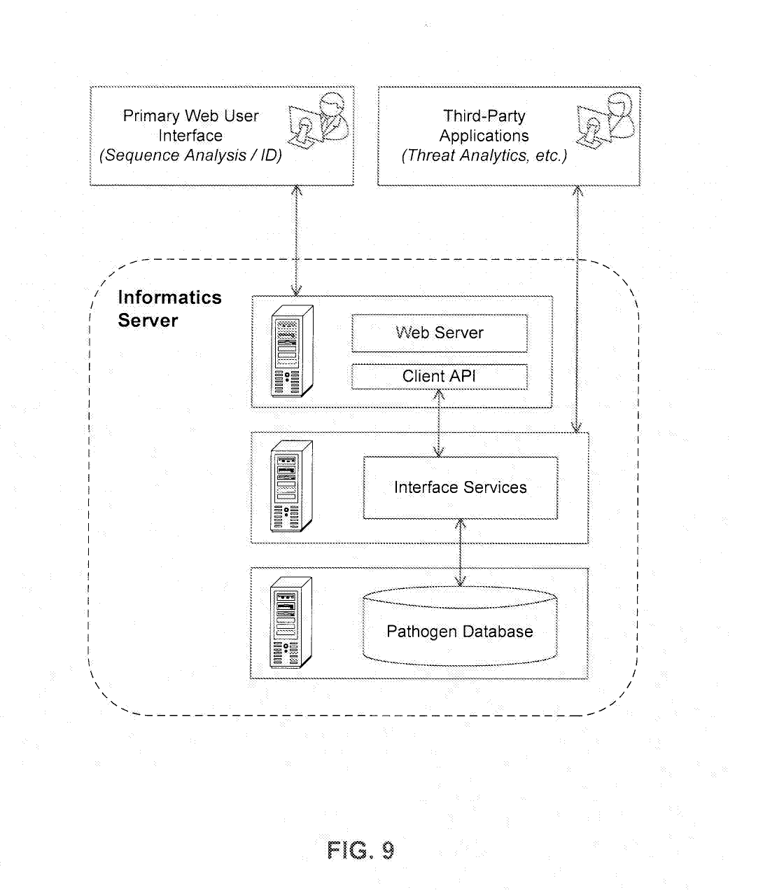

FIG. 9 illustrates an architecture design for the system of the invention. The system includes hardware and software such as servers, data bases, data storage apparatus, interface services, web services, client interfaces, operating systems, and analytics software.

### EXAMPLE II

### Synthesis of Peptide-RNA Conjugate

In an embodiment of the invention, the nucleic acid therapeutic is RNA. Furthermore, the therapeutic RNA may be conjugated to a peptide for bacterial cell permeation and/or for mammalian cell targeting. RNA is a powerful tool for regulatory processes and inhibition of gene expression. The type of RNA to be used in the RANT platform is dependent on what type of pathogen is being treated. For instance, a virus may be more successfully treated by using mammalian intracellular pathways responsive to inhibitory RNA (RNAi) via siRNA to stop viral replication. In the case of bacteria, the RNA must be transported inside of the bacterium to be effective. RNAi is not well understood in bacteria, therefore, antisense inhibition is utilized.

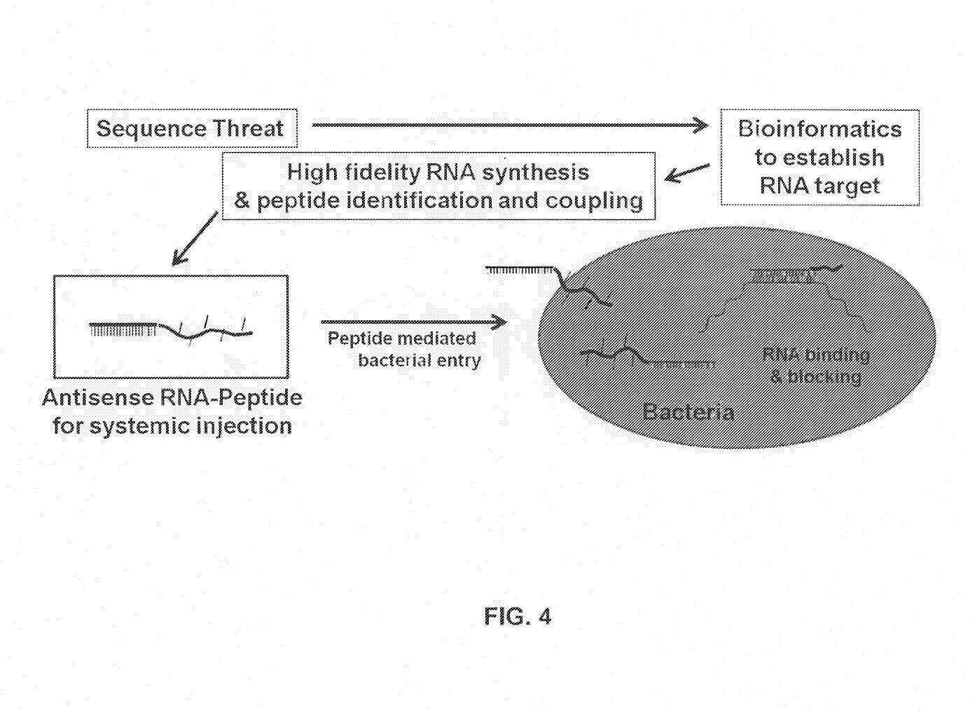

RNA is a regulator of bacterial virulence, as discussed by Gripenland et al. Nat. Rev. 2010, 8, 857-866. Regulatory RNAs including 5′ and 3′ untranslated regions adjacent to coding sequence, cis-acting antisense RNA and trans-acting small coding RNA all influence protein expression and function. A schematic of how peptide-RNA is used to treat bacteria is shown in FIG. 4. In this scenario, a cell penetrating peptide (CPP) is conjugated to antisense RNA. The peptide moiety of the conjugated RNA increases bacterial cell uptake of the therapeutic polynucleotide.

Although direct dosing with naked RNA has been used to knockdown pathogenesis of superbugs like MRSA in culture, a significant barrier for nucleic acid therapies is the bacterial cell wall, as discussed by Yanagihara et al J. Antimicrobial Chemotherapy 2006, 57, 122-126. To overcome the cell wall barrier, peptides derived from bacterial-infecting organisms such as bacteriophages and viruses that can penetrate these bacterial cell walls are attached to RNA. Several peptides with this function have been identified and studied (Fernandez-Lopez et al. Nature, 2001, 412, 452-455; and Turner et al. Blood Cells Mol Dis. 2009, 38, 1-7).

RNA sequences identified and optimized by the bioinformatics component of RANT are synthesized using high-fidelity RNA synthesizers, such as the RNA synthesizer made by NEO-Bio Group, Cambridge, Mass. The RNA is coupled to peptides which permit permeation of bacterial membranes and RNA entry. In the present invention, solid state synthetic methodology for peptide-RNA coupling is employed, although other methodologies may be used.

As shown in FIG. 4, the RANT platform delivers polynucleotides that treat blood bacterial infections (septicemia) and extracellular infections with synthetic peptide-RNA conjugates. The conjugated peptides in these instances are bacterial targeting and cell membrane permeation peptides.

In another embodiment of the invention, the therapeutic nucleic acid is DNA or modified-DNA. These polymers may also be coupled to targeting peptides and/or cell permeating peptides as encompassed by the invention. While targeting and cell permeation peptides are valuable tools for increasing specificity of therapeutic action and reducing toxicity, nucleic acid-peptide coupling is optional.

### EXAMPLE III

### Particle Formation

Material properties of the therapeutic composition of the invention are demonstrated to form nanoparticle polyplexes capable of transporting nucleic acids with stability in serum. The polyplex compositions comprise a synthetic delivery polymer and biologically active compound associated with one another in the form of particles having an average diameter of less than about 500 nm, such as about 300 nm, or about 200 nm, preferably less than about 150 nm, such as 100 nm.

Traditional means of carrying DNA into cells rely on cell membrane disruption such as electroporation or infection with viruses. Both of these have the drawback of not being conducive to rapid high throughput screening of multiple DNA constructs. Nor are they conducive to large scale protein production because electroporation doesn't scale to larger formats and the presence of a virus complicates protein purification and safety issues. Chemical transfection methods provide a more favorable alternative to electroporation and viruses. Lipid-based delivery systems, which encapsulate a deliverable gene into a liposomal structure and enter the cell via membrane disruption/diffusion and active uptake are encompassed by the present invention.

Other polymers, such as poly-L-lysine (PLL), polyethyleneimine (PEI), chitosan, and their derivatives are also encompassed by the invention. Nucleic acid delivery using these compounds relies on complexation driven by electrostatic interactions between the gene and the polycationic delivery agent. Polymer-DNA complexes condense into particles on the order of 60-120 nm in diameter, a size suitable for active endocytosis by mammalian cells. Polymers such as linear PEI and PLL have high transfection rates in a variety of primary cells both in vitro and in vivo. Toxicity is sometimes associated with these polymers due to the high membrane-disrupting charge they carry.

In vivo nucleic acid delivery has size constraints requiring a sufficiently small polyplex to enable long circulation times and cellular uptake. In addition, polyplexes must resist salt and serum induced aggregation. Serum stability is generally associated with a particle size of sub-150 nm hydrodynamic radius maintainable for 24 h. The nanoparticles of the invention, which comprise nucleic acid therapeutic and delivery polymer, have the hydrodynamic radius and material properties for serum stability. In particular, the delivery polymer, when combined with the nucleic acid protects the therapeutic cargo under physiological conditions. The delivery polymers are designed to have characteristics of spontaneous self-assembly into nanoparticles when combined with polynucleotides in solution. The same delivery polymers include design characteristics that allow pH regulated polynucleotide release inside the cell after cellular uptake. In other words, the polymer safely delivers the polynucleotide to the interior of the cell and then releases the therapeutic according to pH in cellular compartments.

Synthetic polymers of the invention that form nanoparticles are based on highly-controlled radical polymerizations of block copolymers. The polymers are pH responsive to promote endolytic release while being capable of prolonged systemic circulation. These polymers provide low-toxicity cell transfection via ionic properties of the nanoparticle. In particular, phosphonium containing monomers also allow the synthesis of a wide variety of copolymers to control charge density, DNA binding affinity, cytotoxicity, and transfection.

Systemic Delivery Platform Design and Rational:

For blood pool infections such as MRSA, the pathogen is largely extracellular and does not require mammalian cell entry for therapeutic efficacy. However, viral pathogens, and some bacteria used in engineered biological warfare, like Bruscella, are intracellular, and have an additional barrier of mammalian cell entry to be efficacious. In this scenario, a delivery particle that circulates in the bloodstream, enters the mammalian cell and then releases therapeutic cargo is an embodiment of the invention.

The RANT system for intracellular pathogens comprises a two-stage delivery process: 1) systemic delivery to the host's tissues and 2) intra-bacterial delivery once inside host's cells. Successful systemic delivery of nucleic acids into mammalian cells is a significant obstacle for all nucleic acid therapies. Polymeric nanoparticles represent a solution to these obstacles. For example, such polymers played a critical role in the first successful demonstration of systemically delivered siRNA in humans via self-assembled nanoparticles (Davis et al. Nature, 2010, 464, 1067-1070). Other delivery technologies exist that are somewhat successful, however many of these are lipid-based technologies and exhibit significant toxicity and off-target effects. Furthermore, currently available delivery agents do not promote high transfection efficiency.

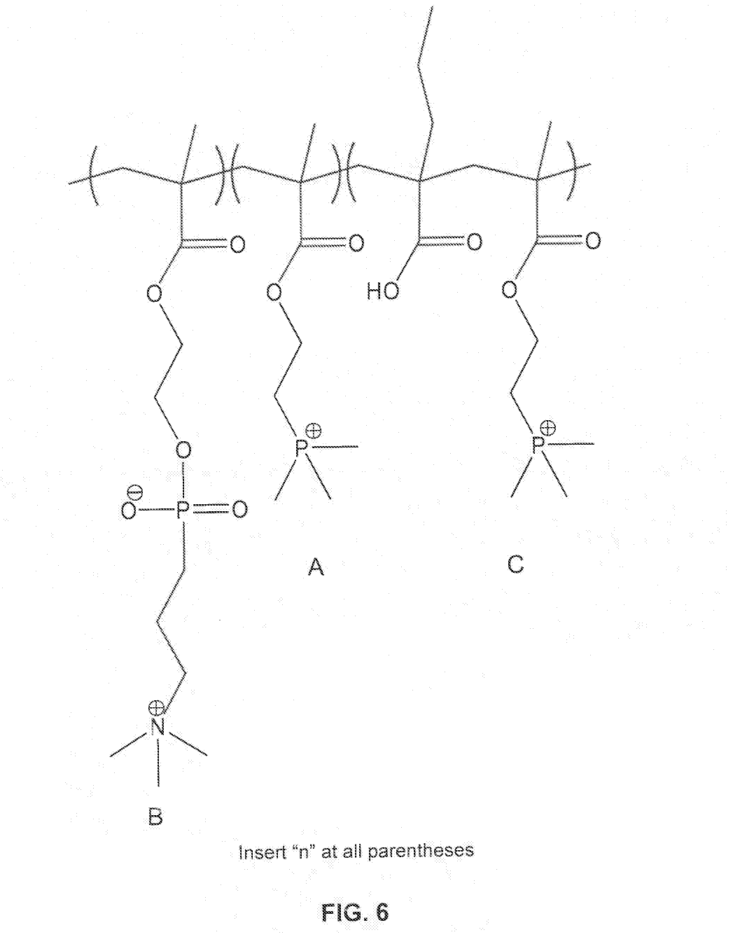

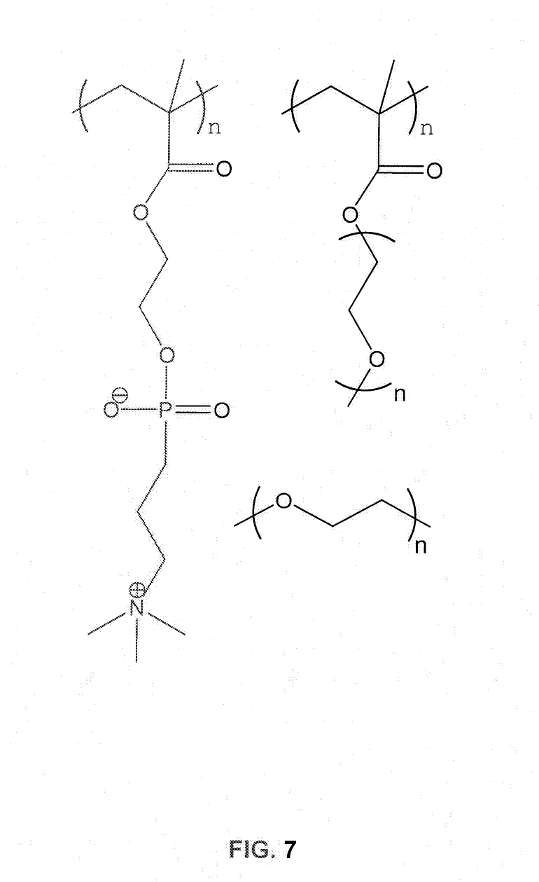

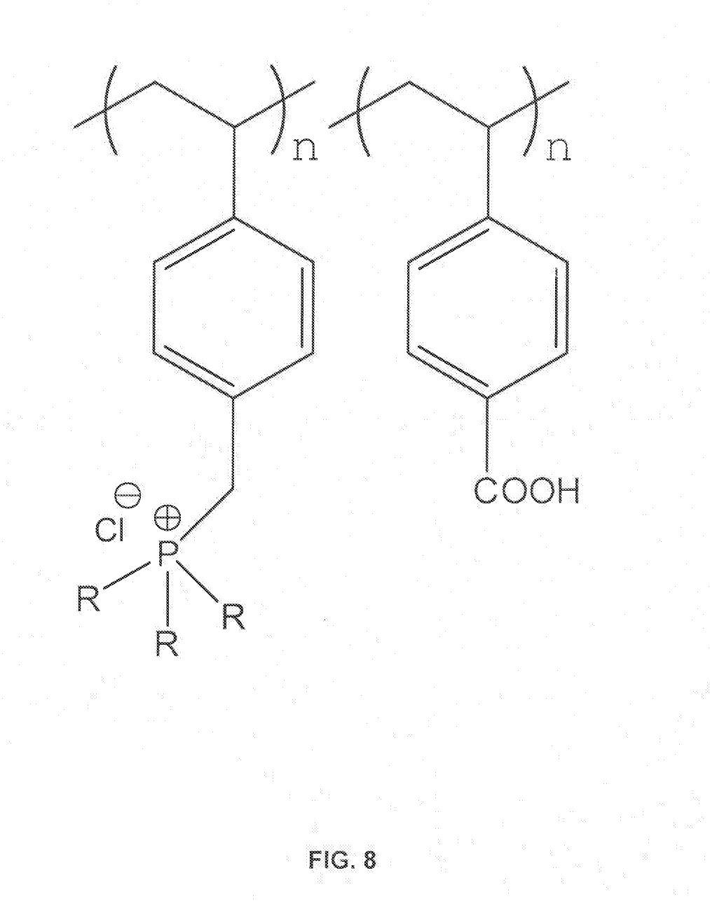

An aspect of the invention is a new family of polymers that overcome the major obstacles of in vivo nucleic acid delivery. These synthetic polymers are disclosed in PCT/US12/42974, incorporated by reference herein. As shown in FIG. 6-FIG. 8, drug delivery compositions comprise ammonium and/or phosphonium containing polymers and/or block copolymers. In an embodiment, the polymers comprise a stabilization block, a complexation block, and an endosomolytic block for non-viral vector gene delivery. Preferred compositions include such copolymers where the complexation block is chosen from polymers of styrenic-based phosphonium containing monomers, such as those shown in FIG. 6, FIG. 7, and FIG. 8. These molecules strongly bind RNA and other nucleic acids, exhibit low cellular toxicity, are serum stable, and provide a better delivery vehicle than the current state of the art.

Current polymeric delivery relies almost exclusively on ammonium moieties to impart positive charge and permit DNA binding. The polymer technology of the invention, however, employs phosphonium moieties for cationic properties at physiological pH values. Structure-property relationships between alkyl substituent lengths on the cationic center are a new feature for this polymer series. These novel vehicles exhibit suitable properties for nucleic acid delivery due to their high affinity for negatively charged nucleic acids. Controlled radical polymerization is employed to enable the synthesis of diblock and higher block order copolymers. Several combinations of the polymeric blocks described herein and other endosomal disruption and serum stability blocks are employed and optimized to meet all of the criteria of successful platform delivery, including: serum stability, formation of particles small enough to avoid liver clearance but large enough to avoid renal clearance, endosomal escape mechanism, protection from nuclease and RNAse enzymatic degradation, and synthetic tailorability. These particles are also useful as wound administration vehicles.

The phosphonium-containing delivery system has several blocks which are responsible for different aspects of transporting peptide-coupled nucleic acids from the systemic compartment to intracellular compartments in tissues, as shown in FIG. 6. The first component, polymeric block A, provides a positive charge for binding and self-assembly with oligonucleotides, such as antisense RNA, DNA and other negatively charged nucleic acids. The second component, block B, provides a steric charge-shielding group which gives the delivery particle stability from assembly with serum proteins. The third component, block C, of the system is a pH-responsive block, to allow endosomal escape after the delivery particle is endocytosed into a cell. This block relies on the acidification of vesicles as they traffic material into cells. Acidification causes protonation of this block and destabilizes the particle for nucleic acid release.

The invention utilizes phosphonium-containing macromolecules for gene delivery. Phosphonium-containing macromolecules can be polystyrene homopolymers or copolymers with variable alkyl substituent lengths attached to the cationic center. Phosphonium vehicles mediate higher gene transfection than ammonium analogs; the longer tributyl alkyl substituent lengths attached to the cationic center also impart enhanced nucleic acid delivery relative to triethyl based analogs.

The invention also utilizes other delivery polymers that form serum-stable nanoparticles. The invention is not limited to the type of delivery polymer and may be adaptable to nucleic acid inhibitor characteristics, such as length, composition, charge, and presence of coupled peptide. The delivery polymer may also be adaptable for material properties of the resultant nanoparticle, such as hydrodynamic radius, stability in the host bloodstream, toxicity to the host, and ability to release cargo inside a host cell. Other polymers useful for delivery include those described in US20090124534 and US20090105115.

### EXAMPLE IV

### Demonstration of Peptide-RNA Conjugate for Extracellular Bacterial Threats

Bulk peptide and RNA synthesis can be carried out by contract manufacturers, such as Neo Group, Inc. (Cambridge, Mass.) using standard methodologies including solid-scaffold protection/deprotection synthesis via high fidelity synthesizers. The peptide-RNA component is the actual therapeutic molecule which enters the pathogen and disrupts its genetic regulation. Referring to FIG. 4, antisense RNA is coupled to a bacterial CPP. In this example, no delivery polymer is present.

Peptides useful in the invention are peptides of diverse origins. Cationic nucleic acid-carrier peptides form productive nanoparticles when mixed with the synthetic polymers of the invention. One example is the peptide KFFKFFKFFK (SEQ ID NO: 1) described in Xie, et al., Molecular Therapy 2004, 10, 652-659. Additional peptides may include TAT peptide and penetratin. The TAT peptide, GRKKRRQRRRPQ (SEQ ID NO: 6), is derived from the transactivator of transcription (TAT) of human immunodeficiency virus and is a cell-penetrating peptide (CPP). Cell-penetrating peptides overcome the lipophilic barrier of cell membranes and deliver large molecules and particles inside the cell for their biological actions. Penetratin™ 1 peptide is a 16-amino acid peptide of sequence RQIKIWFQNRRMKWKK (SEQ ID NO: 7) corresponding to the third helix of the homeodomain of Antennapedia protein. It is able to translocate across biological membranes by an energy-independent mechanism.

CPPs of the invention also encompass cyclic d,l-^{α}peptides, such as, KQRWLWLW (SEQ ID NO: 9), RRKWLWLW (SEQ ID NO: 10), and KKLWLW (SEQ ID NO: 11), as described in Fernandez-Lopez et al. Nature 2001, 412, 452-455. These peptides have antibiotic properties of their own, and also function as carriers of cargo for internal cellular delivery. Additionally, amphipathic peptides LLIILRRRIRKQAHAHSK (SEQ ID NO: 8) and transportin 10 (TP10) AGYLLGKINLKALAALAKKIL (SEQ ID NO: 4), described in Nekhotiaeva et al. FASEB J 2010, 394-396, form productive nanoparticles. Tryptophan rich peptides, such as, Magainin 2 peptide, GIGKWLHSAKKFGKAFVGEIMNS (SEQ ID NO: 3), which was isolated from the African clawed frog (Karas et al, Biochemistry 2002, 41, 10723-31) is another CPP of the platform invention. Furthermore, Indolicidin, ILPWKWPWWPWRR (SEQ ID NO: 5), which was isolated from bovine neutrophils is another CPP encompassed by the present invention. These and other peptides of similar sequence and properties are recognized by one of skill in the art as functional alternatives and are encompassed by the present invention. Furthermore, these peptides may be modified to improve function as necessitated by features of novel pathogens.

The majority of membrane penetrating peptides are hydrophobic. Formation of nanoparticles using hydrophylic polymers facilitates delivery of inhibitory nucleic acids into cells. The synthetic delivery polymers of the invention contain one or more blocks that synergize with properties of the peptide-nucleic acid conjugate. In other words, the delivery polymers are co-designed with the peptide-nucleic acid combination to produce a novel an unexpected increase in transfection efficiency. This combination provides increased efficiency of cargo delivery that is evident in vitro and in whole organisms.

Nucleic acids intended for therapy of known or unknown infectious agents are coupled to penetration peptides using state of the art conjugation methods that employ succinimidyl-6-hydrazinonicotinateacetonehydrazone to succinimidyl-4-formylbenzoate coupling chemistry. This is a specific, well-behaved, and highly efficient conjugation method for peptide-RNA coupling.

In order to covalently couple peptides to nucleic acids, the peptides are prepared for reaction by modifying the N-terminal with a reactive group. In the present invention, the N-terminal of the peptide is modified with S6H (succinimidyl-6-hydrazinonicotinateacetonehydrazone). N-protected peptides are desalted and dissolved in dry DMF. Next, S6H is added in 2× molar excesses to a stirring solution and allowed to react at room temperature for 2 hours. Workup follows procedures known in the art, such as that described by Dirksen et al. J. Am. Chem. Soc. 2006 128, 15602-3.

Similarly, the nucleic acid is prepared for coupling to peptide by modifying bases with succinimidyl-4-formylbenzoate. This is carried out by following published protocol, such as found in Dirksen et al. Other methods of coupling peptides to nucleic acids known in the art may be used.

Demonstration of Peptide-RNA Conjugate for Intracellular Bacterial Threats:

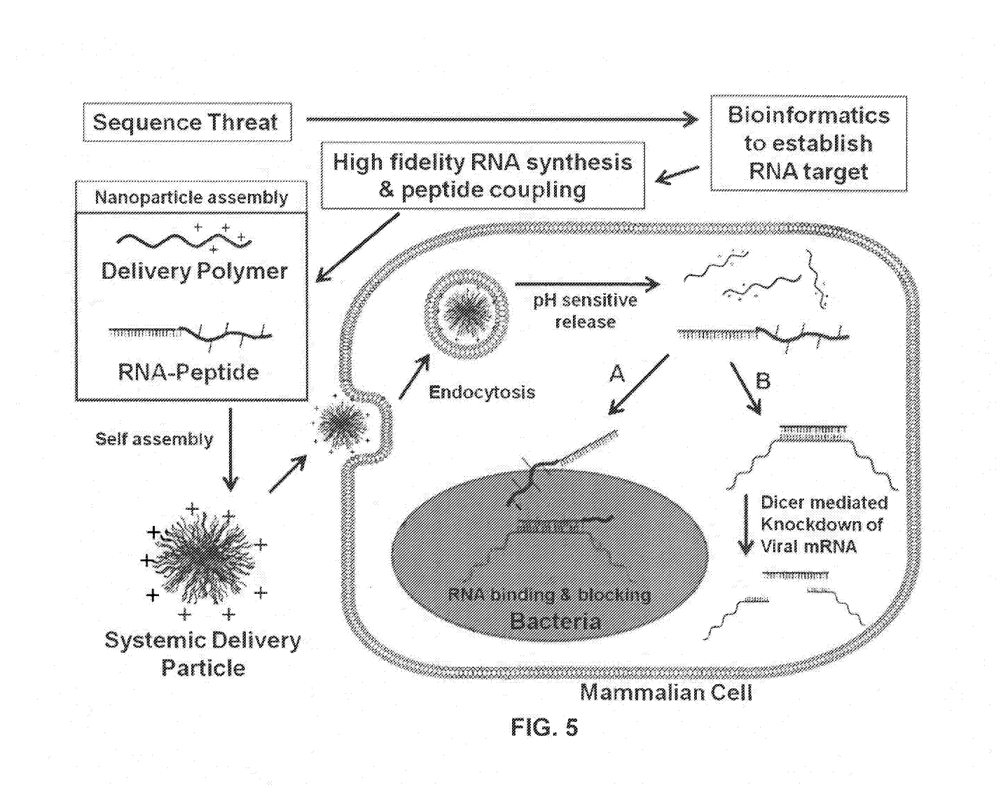

Referring to FIG. 5, a two stage delivery process is shown for intracellular pathogens such as: A) intracellular bacteria and B) viruses. For therapeutic efficacy nucleic acids first enter into mammalian cells and then enter into bacterial pathogens or knock down viral nucleic acids. The first stage involves mixing peptide-RNA conjugate or siRNA with the systemic delivery polymer which self assembles to form a stable particle that releases the RNA once inside the cell. The second stage involves bacteria-penetrating peptide-RNA conjugate entering bacteria inside of cell or siRNA knock down of viral mRNA. Pathogens such as viruses can be knocked down in cell cytoplasm without the need of bacterial penetrating peptides.

Demonstration of RNA Inhibitor for Viral Threats:

Referring to FIG. 2, treatment of disease caused by a viral infection (intracellular) in a host is illustrated. After obtaining sequence information from a known or unknown viral pathogen; designing and synthesizing nucleic acid inhibitor(s) against the viral sequences; and formulating the inhibitor(s) with delivery polymer to form a serum-stable nanoparticle; the host is administered the nanoparticle-containing therapeutic composition. In this example, the nucleic acid inhibitor(s) is siRNA. The siRNA is taken up by the cell and the nucleic acid is released from the nanoparticle once inside the cell.

### EXAMPLE V

### Demonstration of MRSA Inhibition with Peptide-RNA Conjugate

The following example demonstrates effectiveness of the platform enabled therapy in MRSA strains. A statistically significant reduction over selected time period compared to multiple antibiotic controls is achieved. To confirm successful antibacterial nucleic acid agent, peptide-RNA agents are tested against bacteria in culture. Agents are optimized to successfully kill bacteria in culture. The cell culture step establishes a baseline for the second part of the two-stage delivery process of the RANT platform as applied to intracellular infectious agents.

Two strains of MRSA USA 300 are used. MRSA USA 300 is a major source of community-acquired infections in the US Canada, and Europe. Clone FPR3757 is a multidrug-resistant USA 300 strain that is available from ATCC as both the culture (ATCC® BAA-1556™) and genomic DNA (ATCC® BAA-1556D-5). MRSA USA 300 strain is well characterized and all of the unique genes in USA 300 are clustered in novel allotypes of mobile genetic elements (GenBank® CP000255). TCH1516 strain is also available from ATCC as culture (ATCC® BAA-1717™) and genomic DNA (ATCC® BAA-1717D-5) and was also fully sequenced. Target genes include phoB, fmhB, gyrA, and hmrB.

Minimum inhibitory concentration (MIC) analyses are performed as described in Clinical and Laboratory Standards Institute. Methods for Dilution Antimicrobial Susceptibility Tests for Bacteria that Grow Aerobically, 7th ed.; Approved Standard M7-A7; CLSI: Wayne, Pa., USA, 2006; Volume 26, No. 2. Vancomycin and methicillin serve as controls. MIC is determined as the lowest concentration of agent that inhibits bacterial growth detected at A₆₀₀.

Time-kill analyses are performed as described in Haste et al J. Antibiot. 2010, 63, 219-224. Agents at various concentrations are aliquoted into the Falcon tubes. Four-ml of bacteria at 5E5 cfu/ml are added to the tubes. Tubes incubate in a shaker at 37° C., and at 0, 2, 4, 8, 16 and 32 hours are serially diluted with PBS and are plated on tryptic soy agar plates. Colonies are counted after 24 hours at 37° C.

The effect of the human serum on agent activity is determined as described in Haste et al. Marine Drugs 2011 9, 680-689. The MIC in the presence of 20% human serum is performed essentially like the broth microdilution assays except that resazurin is added to a final concentration of 0.675 mg/mL. Plates incubate for 24 h with shaking at 37° C. After incubation, plates are visually assessed for change in color from blue to pink, indicating the resazurin reaction. The MIC in the presence of 20% serum is determined as the lowest agent concentration that did not result in the change in color.

### EXAMPLE VI

### Demonstration of Particle Formation and Stability for Intracellular Threats

Nanoparticle formation and stability are examined by gel electrophoresis shift assay. The ability of the serum stable delivery polymer to bind pDNA and form a polyplex nanoparticle is examined by gel electrophoresis at 60 V. Agarose gels (0.6%, w/v) containing ethidium bromide (0.6 mg/mL) are prepared. Each delivery polymer is dissolved in buffer and is combined with RNA at a multitude of charge ratios to determine optimal binding ratio. The formation of a polymer-RNA complex (polyplex) is determined by a lack of migration of the RNA in the electrophoretic field.

Heparin competitive displacement assay: The optimal charge ratio determined from gel shift assays determines formulation of particle. This optimized polyplex particle is subjected to further stability studies, such as, heparin competition studies to determine the binding strength of the serum stable polymer in the presence of other negatively-charged biomolecules. A series of heparin solutions, for example, 100-1100 mg/mL of heparin ammonium salt from porcine intestinal mucosa (Sigma, St. Louis, Mo.) are prepared. The polyplex solutions are incubated with 10 ml of each heparin concentration for 15 min and subjected to electrophoresis, as described above. The degree of heparin displacement is a measure of particle stability.

Salt displacement and swelling assay: Polyplex particle swelling is an unacceptable quality for in vivo use as it can lead to premature RNA release and rapid clearance in the blood stream. The magnitude of swelling is tested by monitoring an intercalating dye's fluorescence at several salt concentrations around physiological conditions. Polyplexes are prepared as described above in the heparin competitive displacement assay. PicoGreen (Molecular Probes, Eugene, Oreg.) solutions are prepared by 200-fold dilution with 10 mM HEPES buffer containing various concentrations of NaCl (0.1-0.4 M). The polyplex solutions are treated with these salt/dye solutions and are subsequently assayed for fluorescence using a plate reader. Increased florescence at physiological pH suggests further optimization of formulation is required.

RNAse protection assay: A successful nucleic acid delivery system provides protection of polynucleotide degradation until reaching the intracellular environment. Experimental procedures used to measure RNAse protection are known in the art (see Gao X., Biochem. 1996, 35, 1027-1036). Serum delivery polymer is complexed with RNA using optimized formula conditions established in previous steps. The complexes are incubated with 5 mL of fetal bovine serum (FBS) for 0, 1, 2, 4, and 8 h. At the end of each incubation, the complexes are treated with sodium dodecyl sulfate (SDS, 10% w/v) to release the RNA from the polymers. These aliquots are subsequently loaded onto a gel and electrophoresed to detect signs of RNA degradation, such as parent band fading, further migration.

Particle Size Measurements via Dynamic Light Scattering (DLS): Particle size plays an important role in determining blood circulation time and clearance. Nanoparticle size is also a predictor of tissue permeation and selectivity (see Bartlett, et al., Proc. Nat. Acad. Sci. USA, 2007, 104, 15549). Polyplex hydrodynamic diameter is measured on a Zetasizer (Nano ZS) DLS instrument (Malvern Instruments, Worcestershire, UK). Optimized particles are formed based on conditions established in the previous steps. Samples are formed in buffered solutions with 10% FBS to simulate systemic administration conditions, and the particle sizes were measured at several time points post formation to determine the salt and serum stability of the polyplexes. Particles ideal for stable vascular circulation are achieved in the range of 30-150 nm. As an additional predictive indicator, zeta-potential is determined for this same data set. A suitable range for systemic use is close to neutral (−10 to 10 mV).

Particle morphology measurements via TEM: Polymer-DNA complexes are prepared via optimized conditions as described above for the DLS. After formation, the polyplexes are diluted and incubated with buffer and FBS as described in the DLS study. Each sample is applied to a 400-mesh carbon-coated grid. Samples are negatively stained with uranyl acetate (2%, w/v) for 90 s and blotted dry with filter paper. TEM images are recorded with a JEOL JEM-1230 TEM operated at 60 kV.

### EXAMPLE VII

### Toxicity Profile of Naked Peptide-RNA and Platform Peptide-RNA Nanoparticle in Cells

This example demonstrates the low toxicity profile of peptide-RNA and platform conjugate across samples of non-infected cells. Cells are chosen from HeLa, CHO, NIH-3T3, HEK-293, MCF-7, COS-7, or other desirable cell type. Greater than 80% viability in selected cells 24-48 after dosing was achieved.

Evaluation of Uptake and Toxicity Profile in Host Cells:

As a positive control, we show the systemic polymer coupled with the peptide-RNA conjugates demonstrate cell entry and are nontoxic to mammalian cells. Furthermore, some RNA-based therapies may rely on knockdown in mammalian cell cytoplasm. This is evaluated using conventional cell assay and cytotoxic determination methods.

Cellular uptake: Cells are cultured using standard methods and plated on 6-well plates at 60% confluence. The polyplexes are prepared using a Cy5-labelled RNA as a marker. The cells are transfected with the polyplex solutions in serum-containing media. Four hours after initial transfection, cells are trypsinized, pelleted, and resuspended in buffer for fluorescent-activated cell sorting (FACS) analysis. Positive fluorescence level is established by visual inspection of the histogram of negative control cells such that less than 1% appear in the positive region.

MTT Assay: Cells are prepared and transfected with the two-stage delivery platform. After 2 days the cells are treated with media containing 0.5 mg/ml 3-(4,5-Dimethylthiazol-2-yl)-2,5-diphenyltetrazolium bromide (MTT). Cells are incubated for an additional 1 h and are washed with PBS. After cell uptake, the samples are lysed in 250 μl DMSO and sample cell lysates and control cells are analyzed via absorbance. Lysate absorbance for MTT activity is an indirect measure of cell viability.

GAPDH Knockdown Assay: This assay is employed as a way of testing the efficacy of RNAi from platform delivered siRNA.

### EXAMPLE VIII

### The Solution Platform

The invention encompasses an integrated system encompassing technologies than span disciplines, such as, informatics, polymer chemistry, and cell biology. Aspects of these technologies have been brought together to form a unified, effective solution to unsolved problems including drug resistance and infectious disease. The invention has advantages over current methods of therapeutic design in that the unification achieved shortens the steps such that drug resistance cannot outpace development, as it does today. Furthermore, the unified end-to-end solution permits rapid response to unknown threats that may occur in the military arena. Referring to FIG. 2, as applied to unknown categories of pathogens, the invention provides concerted solutions to this problem rather than obstacles.

In one embodiment, the invention comprises a bioterror response facility with apparatus including dedicated nucleic acid sequencers, dedicated informatics system comprising hardware and software for analyzing nucleic acid sequences from known and unknown pathogenic agents; dedicated nucleic acid synthesizers for synthesizing polynucleotides that knockdown known or unknown pathogens; dedicated formulation apparatus for making therapeutic nanoparticles; and dedicated storage apparatus for storing doses of therapeutic nanoparticles.

In one embodiment, one or more major US military sites for biomonitoring are utilized as locations for deployment. In this scenario, a centralized facility is established for sequencing, synthesis, and testing in order to keep the operating cost low and the protocols adaptable. The proposed system is fielded in such a way that diagnostics, analysis of threats, and drug output is rapid and directed.

An embodiment of the RANT platform is envisioned to be housed in a single facility for unified end-to-end processing. This embodiment is envisioned to have dedicated nucleic acid sequencers, which feed output into dedicated informatics systems, which analyze known and unknown sequences and design therapeutics. The designed nucleic acid therapeutic(s) are then synthesized by dedicated apparatus, formulated into nanoparticles and prepared for dosing. By putting these steps into a single dedicated workflow, the protocol becomes highly automated and streamlined beyond simple efficiency increases.

An additional embodiment of the invention envisions an “in theater” bioterror response unit. An in theater response unit can be housed in a dedicated pod-like structure, wherein the pod is outfitted with dedicated apparatus of the invention, and can be transported as a whole to a war zone or other zone where soldiers or other personnel may be exposed to pathogenic threats. Customized transportable pod-like laboratory space can be made by a manufacturer called G-Con. Other types of single-housing facilities may be used. It is to be understood that the invention is not limited by the size or location of the apparatus housing. A pod-housed bioterror response unit is just one structural embodiment of the invention. The pods made by G-Con are completely self-contained for mechanical services and provide support and proper environment necessary for classified environments to produce biotherapeutics. Modules can also provide the ultimate flexibility, mobility, and dedicated space for a dedicated equipment. For example, dimensions of Standard Modules are 18′×43′ and provide 450 sq. ft. of working environment. Modules provide 8′ to 10′ of working height depending on the application. Dimensions of Mega Pod Modules are 36′×43′ and provides 900 sq. ft. of working environment. Modules are constructed from a welded aluminum floor and frame. Modules can accommodate large bioprocess equipment. Modules are mobile and equipped with air bearings for placement and alignment in tight spaces. Pods can be connected to water and electricity from exterior jacks. Redundant air handling systems can provide support for ISO 2 up to ISO 7 environmental quality. Modules have standard hook-ups for biotherapeutic grade water, compressed air, clean steam, CO2 and other custom services. Pods are available in a number of BSL levels based on specific needs. GMP quality pods are also available.

FIG. 10 shows an embodiment of the invention where the RANT system is within a single unified housing. In this embodiment, the end-to-end system is in a POD enclosure, wherein the POD is transportable, has inputs for water, electricity, inside environmental controls, lab set-up including but not limited to molecular biology equipment and reagents, at least part of informatics hardware/software, telecom network, nucleic acid synthesis apparatus, formulation apparatus, and storage for dosage forms of RANT therapeutics.

Other embodiments of the invention include remote control of apparatus via telecom networks. In other embodiments, informatics hardware and software are housed separately, but are operatively linked to the POD via telecom.

What is claimed is:

1. A method of providing a therapeutic nucleic acid composition to treat disease in a host caused by a novel pathogenic agent, the method comprising:
(a) Identifying a pathogen as novel, wherein identification comprises obtaining nucleic acid sequence information from the pathogen and analyzing the sequence information with software;
(b) Identifying sequences within said pathogen that cause disease in a host;
(c) Synthesizing one or more nucleic acid inhibitors of one or more of the sequences that cause disease; wherein the nucleic acid inhibitors are orthogonal to host sequences;
(d) Combining the nucleic acid inhibitors with phosphonium ion-containing synthetic polymers to form nanoparticles stable in the bloodstream of the host; wherein the therapeutic nucleic acid composition comprises the nanoparticle; and wherein the therapeutic nucleic acid composition reduces disease caused by the novel pathogenic agent in a host.

2. The method of claim 1, wherein the phosphonium ion-containing synthetic polymer is a block copolymer comprising a pH responsive block for intracellular release of nucleic acid.

3. The method of claim 1, wherein the nucleic acid inhibitor is coupled to a peptide.

4. The method of claim 3, wherein the peptide is a cell penetrating peptide.

5. The method of claim 3, wherein the peptide is a host cell targeting peptide.

6. The method of claim 1, wherein the pathogen is bacterial, viral, protozoan, or parasite.

7. A method of providing a therapeutic nucleic acid composition to treat disease in a host caused by a drug resistant pathogenic agent, the method comprising:
(a) Identifying a drug resistant pathogen, wherein identification comprises obtaining nucleic acid sequence information from the pathogen and analyzing the sequence information with software;
(b) Identifying sequences within said pathogen that cause disease in a host;
(c) Synthesizing one or more nucleic acid inhibitors of one or more the sequences that cause disease; wherein the nucleic acid inhibitors are orthogonal to host sequences;
(d) Combining the nucleic acid inhibitors with phosphonium ion-containing synthetic polymers to form nanoparticles stable in the bloodstream of the host; wherein the therapeutic nucleic acid composition comprises the nanoparticle; and wherein the therapeutic nucleic acid composition reduces disease in the host caused by the pathogenic agent.

8. The method of claim 7, wherein the phosphonium ion-containing synthetic polymer is a block copolymer comprising a pH responsive block for intracellular release of nucleic acid.

9. The method of claim 7, wherein the pathogen has a cell membrane.

10. The method of claim 9, wherein the pathogen is a bacterial pathogen.

11. The method of claim 7, wherein the nucleic acid inhibitor is coupled to a peptide.

12. The method of claim 11, wherein the peptide is a cell penetrating peptide.

13. The method of claim 11, wherein the peptide is a host cell targeting peptide.

14. A method of providing a therapeutic nucleic acid composition to treat disease in a host caused by a drug resistant pathogenic agent, the method comprising:
(a) Identifying a drug resistant pathogen, wherein identification comprises obtaining nucleic acid sequence information from the pathogen and analyzing the sequence information with software;
(b) Identifying sequences within said pathogen that cause drug resistance in a host;
(c) Synthesizing nucleic acid inhibitors of the sequences that cause drug resistance; wherein the nucleic acid inhibitors are orthogonal to host sequences;
(d) Combining the nucleic acid inhibitors with phosphonium ion-containing synthetic polymers to form nanoparticles stable in the bloodstream of the host; wherein the therapeutic nucleic acid composition comprises the nanoparticle; and wherein the therapeutic nucleic acid composition reduces drug resistance caused by the pathogenic agent in a host.

15. A method of providing a therapeutic nucleic acid composition to treat disease caused by a viral pathogen, the method comprising:
(a) Identifying a viral pathogen, wherein identification comprises obtaining nucleic acid sequence information from the pathogen and analyzing the sequence information with software;
(b) Identifying sequences within said pathogen that cause disease in a host;
(c) Synthesizing nucleic acid inhibitors of one or more sequences that cause disease; wherein the nucleic acid inhibitors are orthogonal to host sequences;
(d) Combining the nucleic acid inhibitors with phosphonium ion-containing synthetic polymers to form nanoparticles stable in the bloodstream of a host; wherein the therapeutic nucleic acid composition comprises the nanoparticles; and wherein the therapeutic nucleic acid composition reduces disease caused by the viral pathogen in a host.

16. A bioterrorism response unit comprising:
(a) A dedicated nucleic acid sequencer, wherein the dedicated sequencers feed output to a dedicated informatics system;
(b) A dedicated informatics system comprising hardware and software for analyzing nucleic acid sequences from known and unknown pathogenic agents;
(c) A dedicated nucleic acid synthesizer for synthesizing polynucleotides;
(d) A dedicated formulation apparatus for making therapeutic nanoparticles;
(e) A dedicated storage apparatus for storing doses of therapeutic nanoparticles.

17. The bioterrorism response unit of claim 16, wherein the response unit is mobile.

18. The bioterrorism response unit of claim 16, wherein the response unit is contained within a single housing.
